(19) United States
(12) Patent Application Publication
(10) Pub. No.: US 2008/0097578 A1
(43) Pub. Date: Apr. 24, 2008

(54) Extender Cuff for Branch Vessel

(75) Inventors: David Erickson, Memphis, TN (US); Prema Ganesan, Oakland, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/551,273

(22) Filed: Oct. 20, 2006

(51) Int. Cl. A61F 2/06

(52) U.S. Cl. 623/1.16

(57) ABSTRACT

A modular graft assembly includes a main graft, a lower extender cuff and an upper extender cuff. By rotating and/or telescoping the main graft, the lower extender cuff, and the upper extender cuff relative to one another, variations in the radial and longitudinal positions of branch vessels are readily accommodated by the modular graft assembly. Accordingly, an aneurysm in a main vessel is excluded while at the same time collateral flow to branch vessels is provided. Further, custom fabrication of a graft assembly to accommodate the vessel structure of a particular patient and the associated costs are avoided.

### BACKGROUND OF THE INVENTION

1. Field of the Invention

The present invention relates to an intra-vascular device and method. More particularly, the present invention relates to a device for treatment of luminal, i.e., intra-vascular, diseases.

2. Description of Related Art

A conventional stent-graft typically included a radially expandable reinforcement structure, formed from a plurality of annular stent rings, and a cylindrically shaped graft material defining a lumen to which the stent rings were coupled. Stent-grafts are well known for use in tubular shaped human vascular or other body vessel.

To illustrate, endovascular aneurysmal exclusion is a method of using a stent-graft to exclude pressurized fluid flow from the interior of an aneurysm, thereby reducing the risk of rupture of the aneurysm and the associated invasive surgical intervention. Although the aneurysm is excluded, it is important that the stent-graft does not occlude fluid flow to branch vessels emanating from the main vessel having the aneurysm.

Unfortunately, a vessel structure is by nature tortuous, asymmetrical and, within limits, individually variable. Accordingly, stent-grafts were often custom fabricated, at considerable expense, to accommodate the vessel structure of a particular patient and to attempt to avoid blocking fluid flow to branch vessels. Or bypass operations were performed to connect closely adjacent vessels such as branchiocephalic trunk with the common carotid and subclavian arteries, so that a stent graft could be placed just below the brachiocephalic artery and cover the common carotid and subclavian arteries safely as the pre installed bypass provides a blood supply to those areteries.

### SUMMARY OF THE INVENTION

In accordance with one example, a modular graft assembly includes a main graft, a lower extender cuff and an upper extender cuff. By rotating and/or telescoping the main graft, the lower extender cuff, and the upper extender cuff relative to one another, variations in the radial and longitudinal positions of branch vessels are readily accommodated by the modular graft assembly. Accordingly, an aneurysm in a main vessel is excluded while at the same time collateral flow to branch vessels is provided. Further, custom fabrication of a graft assembly to accommodate the vessel structure of a particular patient and the associated costs are avoided.

Embodiments are best understood by reference to the following detailed description when read in conjunction with the accompanying drawings.

### BRIEF DESCRIPTION OF THE DRAWINGS

FIG. 1 is an exploded perspective view of a modular graft assembly including a main graft, a lower extender cuff, and an upper extender cuff in accordance with one embodiment;

FIG. 2 is a perspective view of the modular graft assembly of FIG. 1 after assembly in accordance with one embodiment; and

FIG. 3 is a perspective view of the modular graft assembly of FIGS. 1 and 2 deployed within a main vessel in accordance with one embodiment.

Common reference numerals are used throughout the drawings and detailed description to indicate like elements.

### DETAILED DESCRIPTION

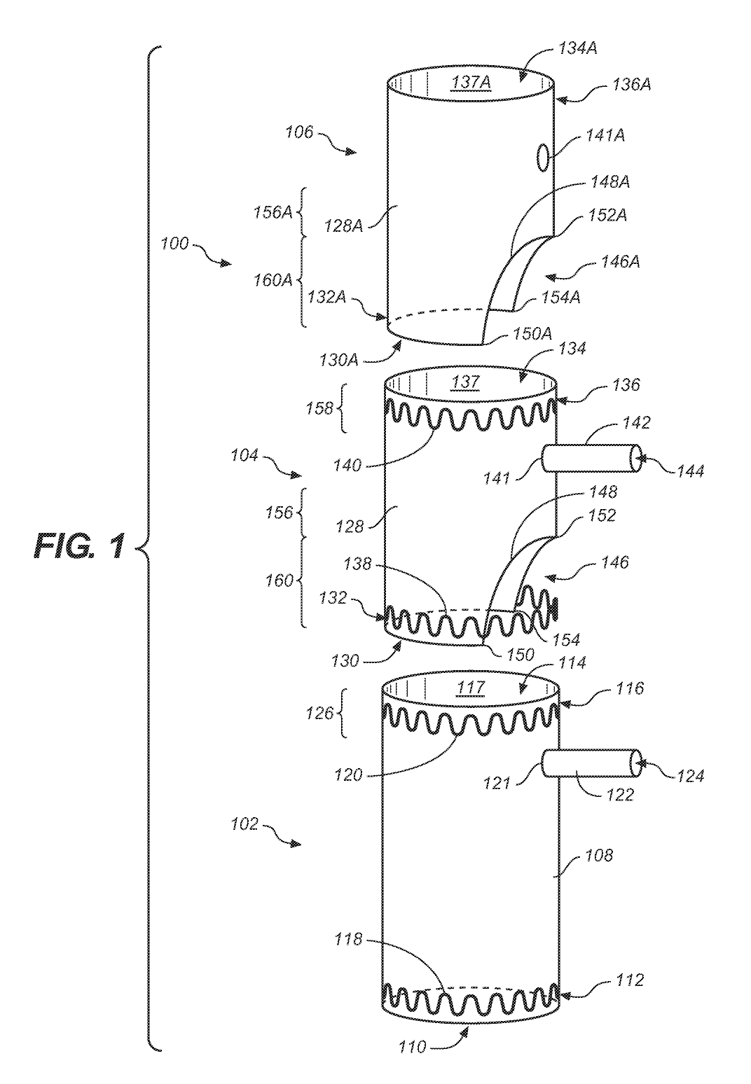

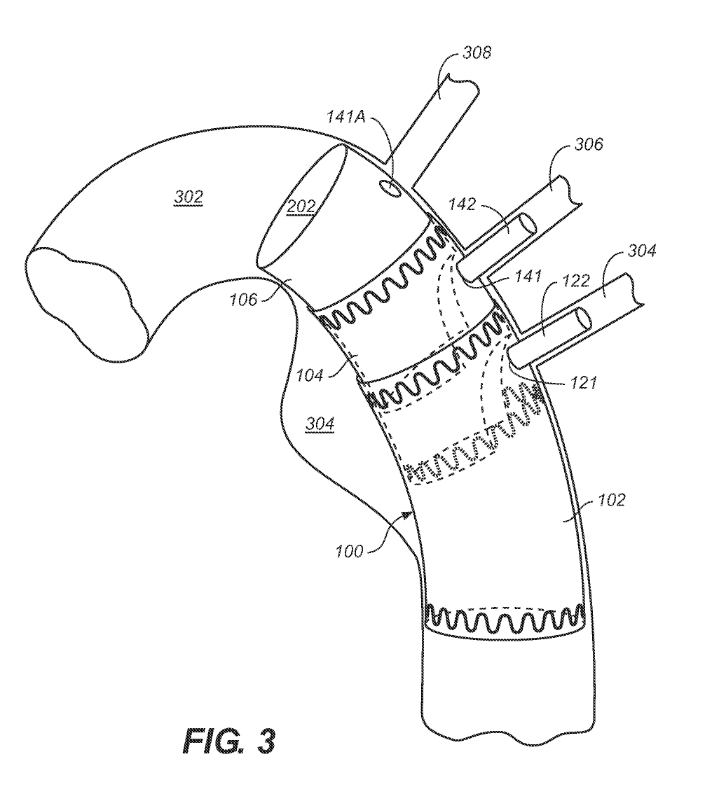

In accordance with one example, referring to FIG. 1, a modular graft assembly 100 includes a main graft 102, a lower extender cuff 104 and an upper extender cuff 106. Referring now to FIG. 3, by rotating and/or telescoping main graft 102, lower extender cuff 104, and upper extender cuff 106 relative to one another, variations in the radial and longitudinal positions of branch vessels 304, 306, 308 are readily accommodated by modular graft assembly 100. Accordingly, an aneurysm 304 is excluded while at the same time collateral flow to branch vessels 304, 306, 308 is provided. Further, custom fabrication of a graft assembly to accommodate the vessel structure of a particular patient and the associated costs are avoided.

More particularly, FIG. 1 is an exploded perspective view of a modular graft assembly 100 including a main graft 102, a lower, e.g., first, extender cuff 104, and an upper, e.g., second, extender cuff 106 in accordance with one embodiment.

Main graft 102 includes a cylindrical graft material 108 having a lower, e.g., first, opening 110 at a lower, e.g., first, end 112. Main graft 102 further includes an upper, e.g., second, opening 114 at an upper, e.g., second, end 116. Main graft 102 defines a lumen 117 extending through main graft 102 from lower opening 110 to upper opening 114.

To facilitate sealing of main graft 102 with the main vessel, main graft 102 further includes a lower, e.g., first, stent ring 118 and an upper, e.g., second, stent ring 120 at lower and upper ends 112, 116, respectively. In one embodiment, stent rings 118, 120 are self-expanding.

Stent rings 118, 120 are optional. In one example, a main graft similar to main graft 102 is formed with either stent ring 118 or stent ring 120, but not both. In another example, a main graft similar to main graft 102 is formed without either of stent rings 118, 120. It yet another example, a main graft similar to main graft 102 is formed with more than two stent rings or other resilient self-expanding structure.

Protruding radially outward from a collateral opening 121 in the cylindrical surface of graft material 108 of main graft 102 is a main graft branch limb 122. In various examples, main graft branch limb 122 is a graft, a stent, or a stent-graft.

Collateral opening 121 and thus main graft branch limb 122 are located not right at the ends, but inward from the ends at a distance from lower and upper ends 112, 116. Main graft branch limb 122 includes a lumen 124 extending through main graft branch limb 122. Lumen 124 is in fluid communication with lumen 117 of graft material 108. As discussed further below in reference to FIG. 3, main graft branch limb 122 supports collateral fluid flow to a branch vessel emanating from a main vessel. However, in another example, a main graft similar to main graft 102 is formed without main graft branch limb 122 and collateral opening 121 supports collateral fluid flow to the branch vessel emanating from the main vessel.

Lower extender cuff 104, sometimes called a thoracic extender cuff, includes a cylindrical graft material 128 having a lower, e.g., first, opening 130 at a lower, e.g., first, end 132. Lower extender cuff 104 further includes an upper, e.g., second, opening 134 at an upper, e.g., second, end 136. Lower extender cuff 104 defines a lumen 137 extending through lower extender cuff 104 from lower opening 130 to upper opening 134.

To facilitate sealing of graft material 128 of lower extender cuff 104 with the main vessel and/or main graft 102, lower extender cuff 104 further includes a lower, e.g., first, stent ring 138 and an upper, e.g., second, stent ring 140 at lower and upper ends 132, 136, respectively. In one embodiment, stent rings 138, 140 are self-expanding.

Stent rings 138,140 are optional. In one example, an extender cuff similar to lower extender cuff 104 is formed with either stent ring 138 or stent ring 140, but not both. In another example, an extender cuff similar to lower extender cuff 104, e.g., extender cuff 106, is formed without either of stent rings 138, 140. It yet another example, an extender cuff similar to lower extender cuff 104 is formed with more than two stent rings or other resilient self-expanding structure.

Protruding radially outward from a collateral opening 141 in the cylindrical surface of graft material 128 of lower extender cuff 104 is an extender cuff branch limb 142. In various examples, extender cuff branch limb 142 is a graft, a stent, or a stent-graft.

Collateral opening 141 and thus extender cuff branch limb 142 are located between and at a distance from lower and upper ends 132, 136. Extender cuff branch limb 142 includes a lumen 144 extending through extender cuff branch limb 142. Lumen 144 is in fluid communication with lumen 137 of graft material 128. As discussed further below in reference to FIG. 3, extender cuff branch limb 142 supports collateral fluid flow to a branch vessel emanating from a main vessel. However, in another example, an extender cuff similar to lower extender cuff 104, e.g., extender cuff 106, is formed without extender cuff branch limb 142 and collateral opening 141 supports collateral fluid flow to the branch vessel emanating from the main vessel.

Lower extender cuff 104 further includes a cutout 146 formed at lower end 132. Cutout 146 is sometimes called a 3-sided window, opening, or scallop of graft material 128.

Cutout 146 is defined by a U-shaped edge 148 of graft material 128. A first end 150 of edge 148 is at lower end 132. Edge 148 extends longitudinally back from first end 150 to an apex 152 (the furthest point or line longitudinally back from the edge) and then back toward a second end 154 of edge 148. Second end 154 of edge 148 is also at lower end 132. Although a particular shape for cutout 146 is described, in other examples, cutout 146 is formed in another shape such as a square, rectangle, triangle, curve or other shape.

Cutout 146 has an area defined by edge 148 that is equivalent to the portion of graft material that is absent (in comparison to an element expected conventional geometric end shape that the end would take if the graft material were not absent) to form cutout 146. If the end was configured in a regular geometric shape or pattern such as a square end or and oblique end or a rapid sinusoidal wave pattern. Then the cutout area is the area missing from a normative end geometry or pattern. As discussed in greater detail below, the area of cutout 146 is greater than the area of collateral opening 121 in main graft 102. This allows lower extender cuff 104 to be rotated and/or moved longitudinally (telescoped (have a varying degree of overlap with the adjacent piece)) relative to main graft 102 without blocking collateral opening 121. Stated another way, this allows lower extender cuff 104 to be positioned so that extender cuff branch limb 142 can be positioned in a branch vessel while having a longitudinal length overlapping internally (or externally) the adjacent main body end and the longitudinal position of the collateral opening 121 without obstructing collateral opening 121. The extender cuff can be rotated and/or telescoped relative to main graft 102 while keeping an overlap between collateral opening 121 and cutout 146.

Upper extender cuff 106 is similar to lower extender cuff 104 and only the significant differences between upper extender cuff 106 and lower extender cuff 104 are discussed. More particularly, upper extender cuff 106 includes a cylindrical graft material 128A, a lower opening 130A, a lower end 132A, an upper opening 134A, an upper end 136A, a lumen 137A, a collateral opening 141A, a cutout 146A, an edge 148A, an end 150A, an apex 152A, and an end 154A similar to cylindrical graft material 128, lower opening 130, lower end 132, upper opening 134, upper end 136, lumen 137, collateral opening 141, cutout 146, edge 148, end 150, apex 152, and end 154 of lower extender cuff 104, respectively.

As discussed in greater detail below, the area of cutout 146A in upper extender cuff 106 is greater than the area of collateral opening 141 in lower extender cuff 104. This allows upper extender cuff 106 to be rotated and/or telescoped relative to lower extender cuff 104 without blocking collateral opening 141. Stated another way, this allows upper extender cuff 106 to be rotated and/or telescoped relative to lower extender cuff 104 while keeping an overlap between collateral opening 141 and cutout 146A.

Although modular graft assembly 100 is set forth above as including both lower extender cuff 104 and upper extender cuff 106, in another example, a modular graft assembly similar to modular graft assembly 100 includes only lower extender cuff 104 or upper extender cuff 106, but not both. In another example, a modular graft assembly similar to modular graft assembly 100 includes two or more lower extender cuffs 104, two or more upper extender cuffs 106, or other combinations of lower extender cuff 104 and upper extender cuff 106.

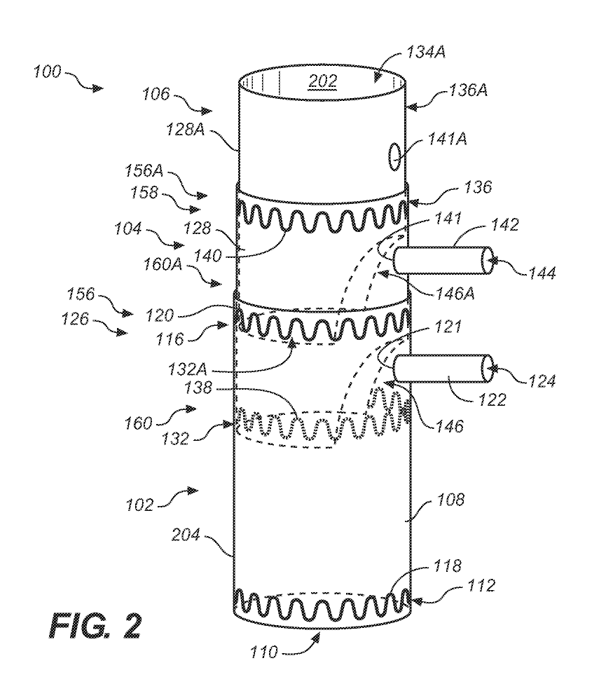

FIG. 2 is a perspective view of modular graft assembly 100 of FIG. 1 after assembly in accordance with one embodiment. Referring now FIGS. 1 and 2 together, a method of assembling modular graft assembly 100 includes inserting lower end 132 of lower extender cuff 104 into upper opening 114 of main graft 102. More generally, lower extender cuff 104 is frictionally connected to main graft 102 by inserting lower extender cuff 104 into main graft 102 such that cutout 146 is aligned with and overlaps collateral opening 121 in main graft 102.

As discussed above, the area of cutout 146 is greater than the area of collateral opening 121. More particularly, the width of cutout 146 is greater than the width of collateral opening 121. This allows lower extender cuff 104 to be rotated relative to main graft 102 while still maintaining an overlap between cutout 146 and collateral opening 121. In this manner, flexibility in the rotational placement of collateral opening 141/extender cuff branch limb 142 relative to collateral opening 121/main graft branch limb 122 is provided. Stated another way, the rotational position of collateral opening 141/extender cuff branch limb 142 relative to collateral opening 121/main graft branch limb 122 is set by rotation of lower extender cuff 104 relative to main graft 102.

Further, the length of cutout 146 is greater than the length of collateral opening 121. This allows lower extender cuff 104 to be longitudinally moved, sometimes called telescoped, relative to main graft 102 while still maintaining an overlap between cutout 146 and collateral opening 121. In this manner, flexibility in the longitudinal placement of collateral opening 141/extender cuff branch limb 142 relative to collateral opening 121/main graft branch limb 122 is provided. Stated another way, the longitudinal position of collateral opening 141/extender cuff branch limb 142 relative to collateral opening 121/main graft branch limb 122 is set by telescoping of lower extender cuff 104 relative to main graft 102.

Similarly, lower end 132A of upper extender cuff 106 is inserted into upper opening 134 of lower extender cuff 104. More generally, upper extender cuff 106 is inserted into lower extender cuff 104 such that cutout 146A is aligned with and overlaps collateral opening 141 in lower extender cuff 104.

As discussed above, the area of cutout 146A is greater than the area of collateral opening 141. More particularly, the width of cutout 146A is greater than the width of collateral opening 141. This allows upper extender cuff 106 to be rotated relative to lower extender cuff 104 while still maintaining an overlap between cutout 146A and collateral opening 141. In this manner, flexibility in the rotational placement of collateral opening 141A of upper extender cuff 106 relative to collateral opening 141/extender cuff branch limb 142 of lower extender cuff 104 is provided.

Further, the length of cutout 146A is greater than the length of collateral opening 141. This allows upper extender cuff 106 to be longitudinally moved, sometimes called telescoped, relative to lower extender cuff 104 while still maintaining an overlap between cutout 146A and collateral opening 141. In this manner, flexibility in the longitudinal placement of collateral opening 141A of upper extender cuff 106 relative to collateral opening 141/extender cuff branch limb 142 of lower extender cuff 104 is provided.

In another example, instead of inserting lower extender cuff 104 into main graft 102, main graft 102 is inserted into lower extender cuff 104 such that cutout 146 is aligned with and overlaps collateral opening 121 in main graft 102. In yet another example, instead of inserting upper extender cuff 106 into lower extender cuff 104, lower extender cuff 104 is inserted into upper extender cuff 106 such that cutout 146A is aligned with and overlaps collateral opening 141 in lower extender cuff 104.

Further, graft material 128 of lower extender cuff 104 includes a middle sealing portion 156. Middle sealing portion 156 is a cylindrical surface longitudinally located between cutout 146 and collateral opening 141.

Further, graft material 108 of main graft 102 includes an end sealing portion 126 at upper end 116. End sealing portion 126 is a cylindrical surface located at upper end 116.

As shown in FIG. 2, middle sealing portion 156 of lower extender cuff 104 overlaps, contacts and seals against end sealing portion 126 of main graft 102. Further, a cutout portion 160 of graft material 128 of lower extender cuff 104 including lower stent ring 138 overlaps, contacts and seals against graft material 108 of main graft 102 thus enhancing the frictional connection and seal between lower extender cuff 104 and main graft 102. Cutout portion 160 is the portion of graft material 128 in which cutout 146 is made.

Similarly, graft material 128A of upper extender cuff 106 includes a middle sealing portion 156A. Middle sealing portion 156A is a cylindrical surface longitudinally located between cutout 146A and collateral opening 141A.

Further, graft material 128 of lower extender cuff 104 includes an end sealing portion 158 at upper end 136. End sealing portion 158 is a cylindrical surface located at upper end 136.

As shown in FIG. 2, middle sealing portion 156A of upper extender cuff 106 overlaps, contacts and seals against end sealing portion 158 of lower extender cuff 104. Further, a cutout portion 160A of graft material 128A of upper extender cuff 106 overlaps, contacts and seals against graft material 128 of lower extender cuff 104 thus enhancing the frictional connection and seal between upper extender cuff 106 and lower extender cuff 104. Cutout portion 160A is the portion of graft material 128A in which cutout 146A is made.

Accordingly, lumens 117, 137, 137A of main graft 102, lower extender cuff 104, upper extender cuff 106, respectively, collectively form a single lumen 202 longitudinally extending through modular graft assembly 100. Upper opening 134A of upper extender cuff 106 forms an upper lumen opening for lumen 202 and lower opening 110 of main graft 102 forms a lower lumen opening for lumen 202. Further, collateral openings 121, 141, 141A of main graft 102, lower extender cuff 104, upper extender cuff 106, respectively, are in fluid communication with lumen 202.

Specifically, collateral opening 121 of main graft 102 is in fluid communication with lumen 202 through cutout 146 of lower extender cuff 104. Accordingly, collateral opening 121 forms a first collateral opening in the cylindrical surface 204 of modular graft assembly 100 collectively created by graft material 108, graft material 128, and graft material 128A of main graft 102, lower extender cuff 104, upper extender cuff 106, respectively.

Similarly, collateral opening 141 of lower extender cuff 104 is in fluid communication with lumen 202 through cutout 146A of upper extender cuff 106. Accordingly, collateral opening 141 forms a second collateral opening in cylindrical surface 204 of modular graft assembly 100.

Finally, collateral opening 141A of upper extender cuff 106 is in directing fluid communication with lumen 202. Collateral opening 141A of upper extender cuff 106 forms a third collateral opening in cylindrical surface 204 of modular graft assembly 100.

FIG. 3 is a perspective view of modular graft assembly 100 of FIGS. 1 and 2 deployed within a main vessel 302 in accordance with one embodiment. Modular graft assembly 100 is deployed into main vessel 302 using any one of a number of well known techniques, i.e., progressive deployment of elements where there is initial deployment of the branch element into the branch and subsequent deployment of the main vessel portion, initial partial expansion/deployment of the main cuff body to a configuration where the main cuff body can be rotated and longitudinally adjusted so that the branch opening and graft are aligned as well as they can be with the location of the branch vessel into which they are to be implanted in the surrounding vessel, or a variation and combination of the such techniques known to those of skill in the art, the particular delivery/deployment technique used is not essential.

Referring now to FIG. 3, main vessel 302, e.g., the aorta, includes an aneurysm 304. Modular graft assembly 100 seals against main vessel 302 above and below, e.g., proximally and distally, aneurysm 304. Accordingly, fluid flows through lumen 202 of modular graft assembly 100 thus bypassing and excluding aneurysm 304. In another example, modular graft assembly is used to stent main vessel 302.

Branching off main vessel 302 are three branch vessels 304, 306, 308, e.g., the subclavian, the common carotid, and the brachiocephalic trunk.

Collateral opening 121 of main graft 102 is aligned with branch vessel 304. Further, main graft branch limb 122 of main graft 102 extends into branch vessel 304. Accordingly, collateral flow from lumen 202 of modular graft assembly 100 to branch vessel 304 is provided through collateral opening 121 and main graft branch limb 122. Illustratively, main graft branch limb 122 stents branch vessel 304.

Similarly, collateral opening 141 of lower extender cuff 104 is aligned with branch vessel 306. Further, extender cuff branch limb 142 of lower extender cuff 104 extends into branch vessel 306. Accordingly, collateral flow from lumen 202 of modular graft assembly 100 to branch vessel 306 is provided through collateral opening 141 and extender cuff branch limb 142. Illustratively, extender cuff branch limb 142 stents branch vessel 306.

Finally, collateral opening 141A of upper extender cuff 106 is aligned with branch vessel 308. Accordingly, collateral flow from lumen 202 of modular graft assembly 100 to branch vessel 308 is provided through collateral opening 141A.

Further, as described above, by rotating and/or telescoping main graft 102, lower extender cuff 104, and upper extender cuff 106 relative to the one previously placed during the deployment process, variations in the radial and longitudinal positions of branch vessels 304, 306, 308 are readily accommodated by modular graft assembly 100. Accordingly, aneurysm 304 is excluded while at the same time collateral flow to branch vessels 304, 306, 308 is provided. Further, custom fabrication of a graft assembly to accommodate the vessel structure of a particular patient and the associated costs are avoided.

A method of assembling a modular graft assembly includes: frictionally connecting a first extender cuff with a main graft comprising a collateral opening, wherein a cutout of the first extender cuff is aligned with and overlaps the collateral opening of the main graft, the first extender cuff includes a collateral opening, the cutout having a greater area than the collateral opening of the main graft. The method can further include rotating the first extender cuff relative to the main graft to set the rotational position of the collateral opening of the first extender cuff relative to the collateral opening of the main graft. The method can further include telescoping the first extender cuff relative to the main graft to set the longitudinal position of the collateral opening of the first extender cuff relative to the collateral opening of the main graft. The method may further include frictionally connecting a second extender cuff with the first extender cuff, wherein a cutout of the second extender cuff is aligned with and overlaps the collateral opening of the first extender cuff, the second extender cuff comprising a collateral opening, the cutout of the second extender cuff having a greater area than the collateral opening of the first extender cuff.

A further method according to the present invention includes the steps of placing a main graft including an integral branch graft extension in a main vessel at an intersection of the main vessel with a first branch vessel, where the integral branch graft extension is deployed in the first branch vessel and provides fluid communication from said main graft into the branch vessel, and positioning and deploying a branch extender cuff branch with integral branch graft, the branch graft extending into a second branch vessel, the cuff branch having a generally cylindrical main body with a scalloped lower edge to overlap an upper portion of said main graft and surround an opening of said main integral branch graft with the scallop opening positioned in the area of the opening of the integral branch graft to maintain patency for branch flow while providing a seal with said longitudinal portion of said main graft. A further step where a second cuff branch provides and extesion and seal for a immediately adjacent third vessel branching from the main vessel. The cuff branch have openings which provide an opening for the passage of blood to the adjacent branch. The main cuff having the side branch opening may include integral branch graft or stent graft extensions providing an adequate landing zone for sealing into the adjacent branch vessels.

This disclosure provides exemplary embodiments. The scope is not limited by these exemplary embodiments. Numerous variations, whether explicitly provided for by the specification or implied by the specification or not, such as variations in structure, dimension, type of material and manufacturing process may be implemented by one of skill in the art in view of this disclosure.

What is claimed is:

1. A modular graft assembly comprising a first extender cuff, said first extender cuff comprising:
a cylindrical graft material comprising:
a first opening at a first end of said cylindrical graft material;
a second opening at a second end of said cylindrical graft material;
a collateral opening in said cylindrical graft material; and
an edge defining a cutout at said first end of said cylindrical graft material, said edge extending longitudinally back from said first end to an apex, and from said apex back to said first end.

2. The modular graft assembly of claim 1 wherein said edge comprises a first end and a second end at said first end of said cylindrical graft material, said edge extending longitudinally inward from said first end of said edge to said apex of said edge and extending longitudinally outward from said apex to said second end of said edge.

3. The modular graft assembly of claim 1 wherein said first extender cuff further comprises an extender cuff branch limb protruding radially outward from said collateral opening.

4. The modular graft assembly of claim 3 wherein said extender cuff branch limb is selected from the group consisting of a graft, a stent, and a stent-graft.

5. The modular graft assembly of claim 3 wherein said cylindrical graft material defines a lumen, said extender cuff branch limb defining a lumen, said lumen of said extender cuff branch limb being in fluid communication with said lumen of said cylindrical graft material.

6. The modular graft assembly of claim 1 wherein said first extender cuff further comprising a first stent ring at said first end.

7. The modular graft assembly of claim 6 wherein said first extender cuff further comprising a second stent ring at said second end.

8. The modular graft assembly of claim 1 wherein said collateral opening is located longitudinally back and at a distance from said first and second ends.

9. A modular graft assembly comprising:
a main graft comprising a collateral opening; and
a first extender cuff frictionally connected to said main graft, said first extender cuff comprising a cylindrical graft material comprising:
a first opening at a first end of said cylindrical graft material;
a second opening at a second end of said cylindrical graft material;
a collateral opening in said cylindrical graft material; and
an edge defining a cutout at said first end of said cylindrical graft material, said cutout overlapping said collateral opening in said main graft.

10. The modular graft assembly of claim 9 wherein said main graft comprising a cylindrical graft material comprising:
a first opening at a first end of said cylindrical graft material of said main graft; and
a second opening at a second end of said cylindrical graft material of said main graft.

11. The modular graft assembly of claim 10 wherein said collateral opening of said main graft is in said cylindrical graft material of said main graft.

12. The modular graft assembly of claim 9 wherein said main graft further comprises a main graft branch limb protruding radially outward from said collateral opening of said main graft.

13. The modular graft assembly of claim 12 wherein said main graft branch limb is selected from the group consisting of a graft, a stent, and a stent-graft.

14. The modular graft assembly of claim 12 wherein a cylindrical graft material of said main graft defines a lumen, said main graft branch limb defining a lumen, said lumen of said main graft branch limb being in fluid communication with said lumen of said cylindrical graft material of said main graft.

15. The modular graft assembly of claim 9 wherein said cylindrical graft material of said first extender cuff comprises a middle sealing portion and a cylindrical graft material of said main graft comprises an end sealing portion, said middle sealing portion sealing against said end sealing portion.

16. The modular graft assembly of claim 15 wherein said cylindrical graft material of said first extender cuff further comprises a cutout portion, said cutout portion sealing against said cylindrical graft material of said main graft.

17. The modular graft assembly of claim 16 wherein said cutout is in said cutout portion.

18. A modular graft assembly comprising:
a main graft comprising a collateral opening;
a first extender cuff frictionally connected to said main graft, said first extender cuff comprising a cylindrical graft material comprising:
a collateral opening; and
an edge defining a cutout, said cutout overlapping said collateral opening in said main graft; and
a second extender cuff frictionally connected to said first extender cuff, said second extender cuff comprising a cylindrical graft material comprising:
a collateral opening; and
an edge defining a cutout, said cutout of said second extender cuff overlapping said collateral opening in said first extender cuff.

19. A method of assembling a modular graft assembly comprising:
frictionally connecting a first extender cuff with a main graft comprising a collateral opening, wherein a cutout of said first extender cuff is aligned with and overlaps said collateral opening of said main graft, said first extender cuff comprising a collateral opening, said cutout having a greater area than said collateral opening of said main graft.

20. The method of assembling a modular graft assembly of claim 19 further comprising rotating said first extender cuff relative to said main graft to set the rotational position of said collateral opening of said first extender cuff relative to said collateral opening of said main graft.

21. The method of assembling a modular graft assembly of claim 19 further comprising telescoping said first extender cuff relative to said main graft to set the longitudinal position of said collateral opening of said first extender cuff relative to said collateral opening of said main graft.

22. The method of assembling a modular graft assembly of claim 19 further comprising frictionally connecting a second extender cuff with said first extender cuff, wherein a cutout of said second extender cuff is aligned with and overlaps said collateral opening of said first extender cuff, said second extender cuff comprising a collateral opening, said cutout of said second extender cuff having a greater area than said collateral opening of said first extender cuff.
